SEQUENCE LISTING (4 sequences)

SEQ ID NO: 1 - DNA, 22 bp, Artificial
  artificial oligonucleotide for complementary sequence
aaaaaaaaaacctaataacaat

SEQ ID NO: 2 - DNA, 27 bp, artificial
  artificial oligonucleotide for target sequence
ggattattgttaaatattgataaggat

SEQ ID NO: 3 - DNA, 27 bp, artificial
  artificial oligonucleotide for mis-matched target sequence
ggattatcgttaaatattgataaggat

SEQ ID NO: 4 - DNA, 25 bp, artificial
  artificial oligonucleotide for probe sequence
ttataactattcctaaaaaaaaaaa

(19) United States
(12) Patent Application Publication
(10) Pub. No.: US 2006/0057604 A1
(43) Pub. Date: Mar. 16, 2006

(54) Method for electrically detecting oligo-nucleotides with nano-particles

(75) Inventors: Chei-Chiang Chen, Taipei City (TW); Chien-Ying Tsai, Taipei County (TW)

Correspondence Address: BACON & THOMAS, PLLC, 625 SLATERS LANE, FOURTH FLOOR, ALEXANDRIA, VA, 22314, US

(73) Assignee: Thinkfar Nanotechnology Corporation, Taipei City (TW)

(21) Appl. No.: 11/078,314

(22) Filed: Mar. 14, 2005

(30) Foreign Application Priority Data
Mar. 15, 2004 (TW) 093106809
Jun. 18, 2004 (TW) 093117629

(51) Int. Cl. C12Q 1/68

(52) U.S. Cl. 435/6

(57) ABSTRACT

A method for detecting a target oligo-nucleotides includes the following steps: providing a substrate mounted with at least a pair of detecting electrodes separated with a gap; coating a surface activation agent on the substrate; providing a plurality of nano-particles and immobilizing them between the two detecting electrodes on the substrate; providing a plurality of capturing oligo-nucleotides; providing a plurality of target oligo-nucleotides and a plurality of probe oligo-nucleotides in order, wherein a portion of the capturing oligo-nucleotides is complementary to the first portion of the sequence of the target oligo-nucleotides; and a portion of the probe oligo-nucleotides is complementary to the second portion of the sequence of the target oligo-nucleotides; and adding a plurality of nano-particles to the gap between the two detecting electrodes.

### BACKGROUND OF THE INVENTION

1. Field of the Invention

The present invention relates to a method for detecting nucleotides with nano particles and electrodes, and more particularly, to a method for minor DNA or RNA sample sequencing and detecting.

2. Description of Related Art

Biochemical reactions or the detection of human diseases are performed in the molecule level, and the molecules as minor as DNA or RNA have to be purified before the biochemical detection can proceed. Polymerase chain reaction (PCR) is the major equipment currently used to amplify nucleotide molecules, and enlarge samples for detecting with high sensitivity. However, the procedure aforecited raises further tasks before a simple detection can be undertaken, and these will slow the detection process.

A nanoparticle probes with electrical DNA sequencing detector is used to measure electrical characteristics such as resistance values, capability values, current values, frequencies or voltage values, to determine whether the particles or target molecules are attached in the gap between two electrodes and the hybridization of three oligo-nucleotides can be assumed. The equipment can be used to detect the conductivity or the density of nucleotides, oligo-nucleotides or proteins on nano gold particles.

Nevertheless, nano particles and portions of nucleotides have to be pre-treated in the conventional use, and this increases experiment time and further tasks. Therefore, a detection method with high efficiency, simple procedures and involving reasonable time is eagerly sought.

Mirkin et. al. (Science, Mirkin et al., 1997) demonstrated a DNA detection methodology utilizing the optical properties of aggregated oligonucleotide-functionalized gold nanoparticles (AuNPs), and many modifications to this method have been reported, such as immobilizing nano particles between two electrodes directly to improve signals.

A DNA detection method using self-assembly multilayer AuNPs is presented with or without the need of a silver enhancer, which represents an alternative method for rapid genetic disease diagnosis.

### SUMMARY OF THE INVENTION

The present invention provides a method for electrically detecting oligo-nucleotides with nano-particles, by which a small scale of samples can be used for detecting with high sensitivity in a short time, and the present invention also provides a portable device for detecting oligo-nucleotides.

The present invention provides a method for electrically detecting target oligo-nucleotides with nano-particles and comprises the following steps: (a) providing a substrate mounted with at least a pair of detecting electrodes separated with a gap; (b) coating a surface activation agent on the substrate; (c) providing a plurality of nano-particles and immobilizing said nano-particles between the two detecting electrodes on the substrate; (d) providing a plurality of capturing oligo-nucleotides; (e) providing a plurality of target oligo-nucleotides and a plurality of probe oligo-nucleotides in order, wherein a portion of the capturing oligo-nucleotides is complementary to the first portion of the sequence of the target oligo-nucleotides, and a portion of the probe oligo-nucleotides is complementary to the second portion of the sequence of the target oligo-nucleotides; and (f) adding a plurality of nano-particles to the gap between the two detecting electrodes.

The hybridization conditions of capture oligo-nucleotides - target oligo-nucleotides and probe oligo-nucleotides can be predicted and the particles or target molecules immobilized in the gap between two electrodes can be determined by detecting electrical characteristics such as resistance values, capability values, current values, frequencies or voltage values.

The present invention can be selectively performed with heating or addition of a salt solution to increase the sensitivity of detecting. To perform the procedure, first heat the substrate mounted with at least one pair of detecting electrodes, and wash the substrate with water, or add a conductive salt solution into the gap between two detecting electrodes on the substrate and then remove the solution. Further, a reductant can be added into the gap after the conductive salt solution is added, to reduce the metal ions into metal atoms and deposit on nano particles, and thus the sensitivity of detecting will be increased.

The appropriate surface activating agents used in the substrate of the present invention can be any conventional one, but preferably is a thiosilane, and more preferably is a trimethoxysilane such as 3-Mercaptopropyl-trimethoxysilane.

In the present invention, one end of nano particles, capture oligo-nucleotides and probe oligo-nucleotides is preferably linked with a thiol group to increase the attachment force between the oligo-nucleotides fragment and nano particles.

The gap on the substrate between two electrodes is not limited, and preferably is in the range of 250 nm and 5000 nm, and more preferably is in the range of 250 nm and 1000 nm. The material of nano particles can be any conventional material, but preferably is selected from a group consisting of Au, Ag, Pd, Pt, C, Ni, Ti, Cu, Fe and Co, and most preferably is Au. The diameter of particles preferably is nano-meter, and more preferably is less than 300 nm, and most preferably is in the range of 5 nm to 50 nm.

The arrangement of the detecting electrodes mounted on the substrate of the present invention is not limited, and preferably is arranged in a manner of array with a plurality of electrodes. Meanwhile, the amount of the detecting electrodes on the substrate is not limited, and preferably is fewer than 399. The conductive salt solution can be any conventional one, but preferably is silver salt or chlorauride, and the reductant can be any conventional one, but preferably is selected from a group consisting of citrate, tannate, and borate.

The quantity of gold particles between two electrodes and the target oligo-nucleotide can be determined by measuring the conductivity. When the signal increases on the substrate, it means the density of gold particles has risen. When two electrodes separated with nano-meter gap and oligo-nuclotides are immersed into the solution with the gold particles, the gold particles fill the gap between two electrodes.

The present invention provides a method for detecting nucleotides with nano-particles and is performed by detecting the electrical characteristics between said two detecting electrodes, and the electrical characteristics can be any conventional ones, but preferably are resistance values, capability values, current values, frequencies or voltage values.

Other objects, advantages, and novel features of the invention will become more apparent from the following detailed description when taken in conjunction with the accompanying drawings.

### BRIEF DESCRIPTION OF THE DRAWINGS

FIG. 1 shows the procedure of the present invention for detecting oligo-nucleotides;

FIG. 2 (a) shows the FE-SEM image for the gold particles attached into the nano-gap electrode of the monolayer; (b) shows the FE-SEM image for the hybridization of gold particles labeled tDNA with cDNA and pDNA to become a self-assembly multilayer;

FIG. 3 shows the device to measure the electrical characteristics on the substrate;

FIG. 4 (a) shows the current-voltage curves for monolayer of gold particles, scan rate 10 mV/s; (b) shows current-voltage curves for multilayer of gold particles, scan rate 10 mV/s;

FIG. 5 shows the I-V curves of the nano-gap electrode measured by using different concentrations of tDNA: (A) 0.1 μM; (B) 1 nM; (C) 10 pM and (D) 1 fM, tDNA were hybridized to cDNA and pDNA in 0.3 M PBS for 2 hours in all experiments;

FIG. 6 (a) shows the image for multilayer of gold particles after complementary hybridization but before denaturing, (b) is the FE-SEM image of gold particles after denaturing, the concentration of tDNA is 1 nM for hybridization. For denaturing, the substrate was immersed into the 0.3 M NaCl, PBS buffer and was heated to 60° C. for 3 minutes; (c) is a current-voltage curve for double layer of gold particles with complementary hybridization with a scanning rate of 10 mV/s; (d) is a current-voltage curve for gold particles after denaturing;

FIG. 7 (a) shows the FE-SEM image for multilayer of gold particles for single base mismatch tDNA hybridization before denaturing, (b) is the FE-SEM image of gold particles for single base mismatch tDNA hybridization after denaturing, the concentration of tDNA is 1 nM for hybridization. For denaturing, the substrate was immersed into the 0.01 M NaCl and PBS buffer for 2 hours; (c) is a current-voltage curve for multilayer of gold particles with single base mismatch tDNA hybridization with a scanning rate of 10 mV/s; (d) is a current-voltage curve of gold particles layer for single base mismatch tDNA after denaturing.

### DETAILED DESCRIPTION OF THE PREFERRED EMBODIMENT

### EXAMPLE 1

### Substrate Activation and Monolayer of Gold Particles Formation

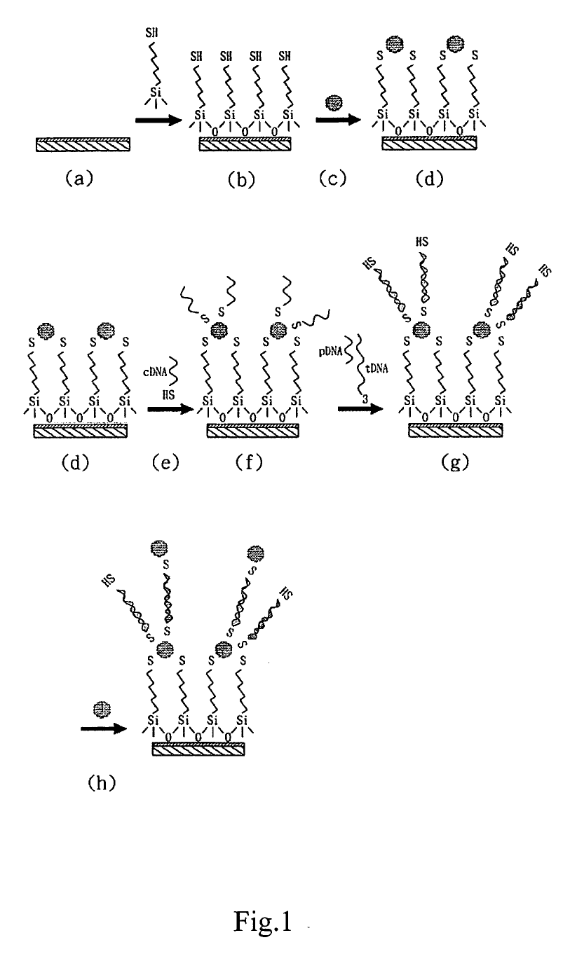

In reference to FIG. 1(a), a substrate mounted with two electrodes is provided, and the gap between the two electrodes is 300-600 nm.

Immerse the substrate into a solution with equal volume of concentrate sulfuric acid and methanol for 30 minutes, and wash the substrate. Rinse the substrate with non-ionic water (over 18 ΩW cm). Immerse the substrate into concentrate sulfuric acid for 5 minutes and wash it with water again. Place the substrate in boiled non-ionic water for several minutes and begin the procedures of substrate activation.

Prepare 1 mM solution of 3-Mercaptopropyl trimethoxysilane (Sigma Chemical Co.) with DMSO. Immerse the substrate into the solution for at least 2 hours at room temperature, rinse the substrate with DMSO and dry in the environment of nitrogen (FIG. 1(b)). Immerse the activated substrate into nano-gold particles solution (AuNPs) for 8-12 hours (FIG. 1(c)), wash the substrate with non-ionic water and dry in the environment of nitrogen gas. An obtained substrate having a monolayer of AuNPs is shown in FIG. 1(d)).

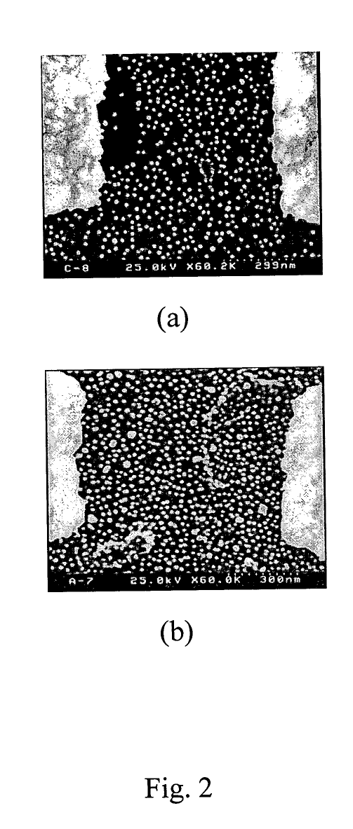

Observe the monolayer of AuNPs on the substrate by Field-Emission Scanning Electron Microscopy (FE-SEM), and the result can be seen in FIG. 2(a). The result indicates the monolayer of AuNPs on the substrate, and the density of AuNPs is about 1200 particles/μm².

### EXAMPLE 2

### Immobilizaton and Hybridizaiton of Oligo-Nucleotides

Four oligo-nucleotide fragments are designed for the example: capture oligonucleotide (cDNA) as seen in SEQ ID NO. 1; target oligonucleotide (tDNA) as seen in SEQ ID NO. 2; probe oligonucleotide (PDNA) as seen in SEQ ID NO. 3; and single base mismatched target oligonucleotide (m-tDNA) as seen in SEQ ID NO. 4. Wherein, cDNA fragment and pDNA fragment both have a portion which is complementary to a different portion of tDNA.

In reference to table 1, four fragments are shown with sequences. Nucleotide bases with underlines indicate a complementary portion, and the single base with frame indicates a single base mutant, which leads to mis-match.

TABLE 1

| Oligo- | |
|---|---|
| nucleotides | Sequence |
| cDNA | 3′-HS-A₁₀-CCT AAT AAC AAT-5′ |
| tDNA | 5′GGA TTA T [symbol] G TTA AAT ATT GAT AAG |
| | GAT-3′ |
| m-tDNA | 5′GGA TTA T [symbol] G TTA AAT ATT GAT AAG |
| | GAT-3′ |
| pDNA | 3′-TTA TAA CTA TTC CTA-A₁₀-SH-5′ |

In reference to FIG. 1(e), 100 μl of 1 μM cDNA is deaerated with pH 6.6 HEPES(4-(2-hydroxyethyl)-1-piperazineethane sulfonic acid, 5 mM EDTA included) 10 mM. Immerse the substrate having the AuNPs monolayer prepared in example 1 into the cDNA solution for 15 hours at room temperature. Wash the substrate with pH6.5 SPSC buffer (contains 50 mM phosphate buffer and 1M NaCl solution) to remove un-bounded oligo-nucleotide molecules, and dry the substrate in the environment of nitrogen (FIG. 1(f)).

Prepare four substrates as described above, immerse them into tDNA solutions with various concentrations of 0.1 μM, 1 nM, 10 pM and 1 fM, and also a pDNA solution with 0.1 μM for 2-hour hybridization. tDNA and pDNA will be linked to the complementary portion of cDNA (FIG. 1(g)). The excess oligo-nucleotide will be removed by immersing the substrate into SPSC buffer. Then immerse the substrate into 0.3 M PBS buffer (0.1M NaCl, 10 mM NaH₂PO₄/Na₂HPO₄, pH7) with AuNPs (FIG. 1(h)). Wash the substrate with 0.3M PBS buffer and dry it in the environment of nitrogen. The substrate is ready for measuring the efficiency of hybridization.

In observation of the substrate by Field-Emission Scanning Electron Microscopy, the result is shown in FIG. 2 (b). AuNPs attach into the nano-gap of electrodes and this indicates that hybridization is successful. The density of AuNPs is about 2900/μm².

To improve the specificity of hybridization, the substrate is treated with 0.01M PBS buffer at room temperature, and then rinsed with 0.3M PBS buffer once before the electrical characteristics are measured. The electrical behavior is measured by a Hewlett Packard, 4156A precision semiconductor parameter analyzer in the −1 to +1 V range with a sweep rate of 1 mV/s.

### EXAMPLE 3

### Results

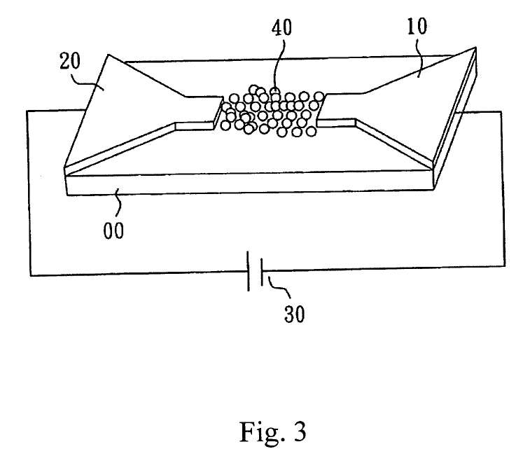

FIG. 3 shows the device to measure the electrical characteristics on the substrate. A source 10, a drain 20 and a voltage generator 30 are on the substrate 00. When the voltage generator 30 provides a voltage, AuNPs 40 with hybridized oligo-nucleotides gather in the nano-gap of two electrodes 10, 20. Because of the conductivity of gold, the changing electrical behavior is measured. Based on the results, when no AuNPs 40 attach to the nano-gap of two electrodes 10, 20, the current measured is less than 50 mA.

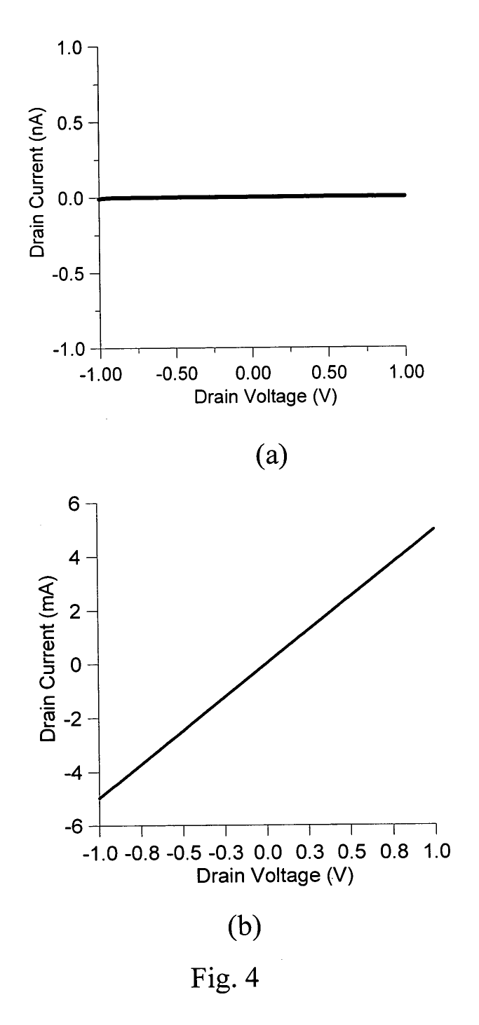

FIG. 4 (a) shows the current-voltage curves for monolayer of gold particles, scan rate 10 mV/s; (b) shows current-voltage curves for multilayer (hybridization successful) of gold particles, scan rate 10 mV/s. Electrons tunnel more readily through the junction when enough energy is supplied. The linear curve is typical of ohmic devices.

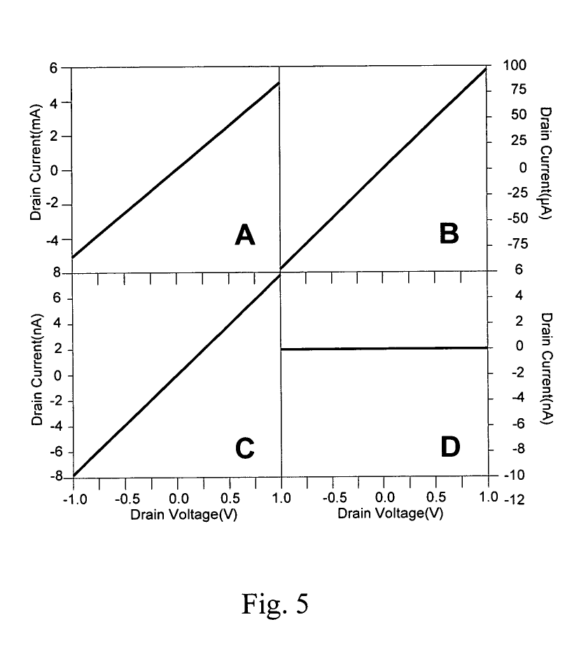

FIG. 5 shows the I-V curves of the nano-gap electrode measured by using four different concentrations of tDNA: (A) 0.1 μM; (B) 1 nM; (C) 10 pM and (D) 1 fM. The result of the current-voltage curve indicates the sensitivity of the present invention is close to 10 pM.

### EXAMPLE 4

### Removal of Hybridized Oligo-Nucleotide

Immerse the substrate after successful hybridization into 0.3M PBS buffer containing NaCl, heat the substrate to 60° C. for 3 minutes, and the oligo-nucleotide hybridized to monolayer AuNPs is removed.

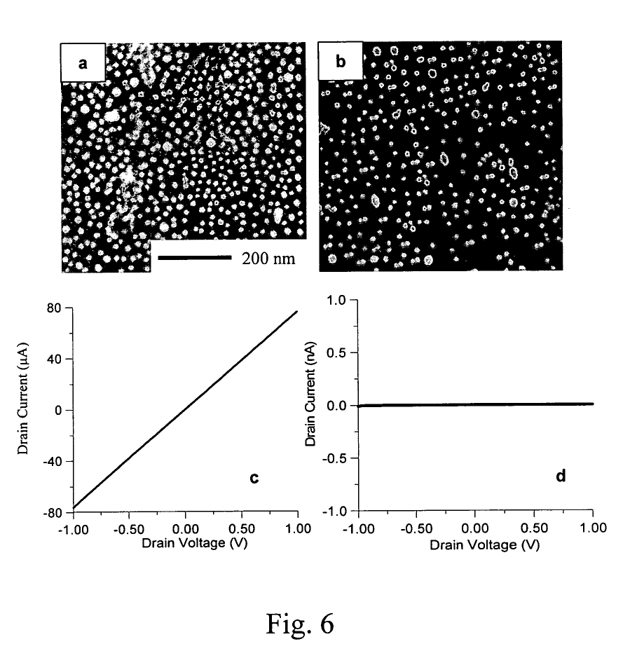

FIG. 6 shows the removing result by FE-SEM and the electrical behavior variation. In FIG. 6(a), high density of AuNPs is observed on the substrate after hybridization, and the corresponding current-voltage curve is shown in FIG. 6(c). After the process of removing hybridized oligo-nucleotides, the density of AuNPs decreases obviously, and the corresponding current-voltage curve measured no signal in FIG. 6(d).

### EXAMPLE 5

### Specificity Test

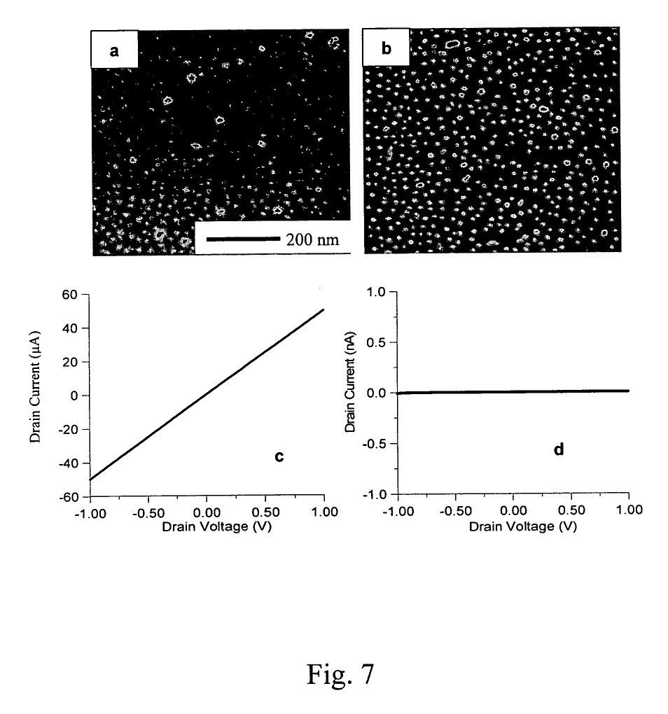

Perform example 2 with 1 nM of single-base mutant m-tDNA, observe the substrate by FE-SEM after hybridization and just before washing, the distribution of AuNPs is as shown in FIG. 7(a), and the current-voltage curve is as seen in FIG. 7(c). Place the substrate into 0.3M PBS buffer containing NaCl for 2 hours, and the distribution of AuNPs can be seen in FIG. 7(b), and the current-voltage curve is FIG. 7(d). According to the data, non-specific oligo-nucleotides are washed off by the high stringency buffer, thus no conspicuous current-voltage signals are measured.

As described above, heating or adding salt solutions into the gap between two electrodes can denature mis-matched tDNA and cDNA, and the non-specific tDNA can be removed by water washing. This decreases the conductivity, and the sensitivity of detection increases. Further, no single base mis-matched tDNA and cDNA can still be complementarily hybridized in the denaturing condition, and the AuNPs are kept in the nano-gap of the electrodes without change of electrical behavior.

In the condition of silver salt being used as a conductive salt, a reductant is further added to reduce the silver ion and the metal will deposit onto the surface of AuNPs, and this improves the conductivity in the nano-gap.

As described previously, the present invention discloses a method for detecting nucleic acids or oligo-nucleotides with high specificity and high sensitivity. The hybridized nucleotides can be removed by appropriate procedure, and the substrate can be re-used to detect the same target oligo-nucleotides. The cost of detection is decreased and the detecting results are reliable in comparison to the prior art.

Although the present invention has been explained in relation to its preferred embodiment, it is to be understood that many other possible modifications and variations can be made without departing from the spirit and scope of the invention as hereinafter claimed.

What is claimed is:

1. A method for electrically detecting target oligo-nucleotides with nano-particles comprises the following steps:
(a) providing a substrate mounted with at least a pair of detecting electrodes separated with a gap;
(b) coating a surface activation agent on the substrate;
(c) providing a plurality of nano-particles and immobilizing said nano-particles between the two detecting electrodes on the substrate;
(d) providing a plurality of capturing oligo-nucleotides;
(e) providing a plurality of target oligo-nucleotides and a plurality of probe oligo-nucleotides in order, wherein a portion of the capturing oligo-nucleotides is complementary to the first portion of the sequence of the target oligo-nucleotides, and a portion of the probe oligo-nucleotides is complementary to the second portion of the sequence of the target oligo-nucleotides; and
(f) adding a plurality of nano-particles to the gap between the two detecting electrodes.

2. The method as claimed in claim 1, further comprising a step (g) after step (f), said step (g) comprises detecting the electrical characteristics between said two detecting electrodes.

3. The method as claimed in claim 2, wherein said electrical characteristics are resistance values, capability values, current values, frequencies and voltage values.

4. The method as claimed in claim 1, wherein the step (f) further includes adding a conductive salt solution between said two detecting electrodes.

5. The method as claimed in claim 4, wherein said conductive salt solution contains silver salts.

6. The method as claimed in claim 4, wherein a reductant is further added between said two detecting electrodes after said conductive salt solution is added.

7. The method as claimed in claim 6, wherein said reductant is selected from the group consisting of: citrate, tannate, and borate.

8. The method as claimed in claim 6, wherein a step (fl) is further included after said step (f), said step (fl) comprises heating said substrate mounted with at least a pair of detecting electrodes separated with a gap, and washing said gap.

9. The method as claimed in claim 1, wherein said surface activation agent is trimethoxysilane.

10. The method as claimed in claim 9, wherein said surface activation agent is 3-Mercaptopropyl-trimethoxysilane.

11. The method as claimed in claim 1, wherein one end of said capture oligo-nucleotides or said probe oligo-nucleotides is linked with a thiol group.

12. The method as claimed in claim 1, wherein said capture oligo-nucleotides are immobilized on said substrate by chemical bonding.

13. The method as claimed in claim 1, wherein said gap between detecting electrodes ranges from 250 nm to 5000 nm.

14. The method as claimed in claim 1, wherein the material of said nano-particles is selected from a group consisting of Au, Ag, Pt, C, Ni, Ti, Cu, Fe and Co.

15. The method as claimed in claim 1, wherein the diameter of said nano-particles is less than 300 nm.

16. The method as claimed in claim 1, wherein said detecting electrodes are formed in a manner of array on said substrate and with at least one pair.

17. The method as claimed in claim 16, wherein said detecting electrodes are fewer than 399 pairs.
